(19) United States
(12) Patent Application Publication
(10) Pub. No.: US 2009/0035234 A1
(43) Pub. Date: Feb. 5, 2009

(54) CONTINUOUS SPRAY SUNSCREEN COMPOSITIONS

(75) Inventors: Corey Cunningham, Larsen, WI (US); Scott W. Wenzel, Neenah, WI (US); Stacy Averic Mundschau, Weyauwega, WI (US)

Correspondence Address: Christopher M. Goff (27839);ARMSTRONG TEASDALE LLP, ONE METROPOLITAN SQUARE, SUITE 2600, ST. LOUIS, MO, 63102, US

(73) Assignee: KIMBERLY-CLARK WORLDWIDE, INC., Neenah, WI (US)

(21) Appl. No.: 11/831,615

(22) Filed: Jul. 31, 2007

(51) Int. Cl. A61K 8/30; A61Q 17/04

(52) U.S. Cl. 424/59; 424/60

(57) ABSTRACT

The present disclosure generally relates to sunscreen compositions that are suitable for topical application to human skin and hair. More particularly, the disclosure relates to sprayable sunscreen compositions having improved water resistance and a low overall percentage of sunscreen actives. To achieve the improved water resistance, a dimethicone derivative is incorporated into the sunscreen compositions.

### BACKGROUND OF DISCLOSURE

The present disclosure generally relates to sunscreen compositions that are suitable for topical application to human skin and hair. More particularly, the disclosure relates to sprayable sunscreen compositions having improved water resistance and a low overall percentage of sunscreen actives. To achieve the improved water resistance, a dimethicone derivative is incorporated into the sunscreen compositions.

The damaging effects of sunlight on human skin have long been noted. In general terms, harmful ultra-violet (UV) rays, particularly those originating from sunlight, which penetrate the upper atmosphere and reach the earth's surface can be classified into two types: (i) high energy UV-B rays (290-320 nm wavelength) which are absorbed just above the dermis and are responsible for sunburn and tanning effects; and (ii) low energy UV-A rays (320-400 nm wavelength) which penetrate deeper into the skin (to the dermis and beyond), and which cause damaging effects that are more long term in nature, such as skin ageing.

Sunscreen compositions or formulations may be applied directly to the skin to combat sunburning of the skin and the numerous damaging effects resulting from exposure to the sun. When a sunscreen formulation, utilizing the proper sun protection factor (SPF, which is a measure of the protection from the sun afforded by a sunscreen agent or composition) is applied uniformly to the body, sunscreens can be highly effective in protecting against sunburn and damage that can lead to photoaging. However, sunscreen failure can occur when areas of the body are missed during application. Furthermore, mild sweating or swimming is often sufficient to remove most commercially available sunscreen formulations from a person's skin, necessitating multiple applications to the skin. The need to repeatedly apply sunscreen is costly to the consumer, inconvenient and tends to cause consumers to delay reapplication of the sunscreen which may lead to sunburn.

For sunscreens intended for beach wear, it is thus important to determine the water resistance properties of the sunscreen to ensure adequate protection for users. To achieve high SPF products, typical sunscreen lotions and creams will incorporate several film forming ingredients to ensure that the active ingredients remain on the skin after exposure to water or perspiration. However, many of these film formers are not available for alcohol based sunscreen spray products due to solubility, instability, or settling that occurs, or interference with the end product functionality (e.g., dispensing). Often, the overall level of film former is also limited by aesthetics as the higher molecular weight polymers tend to give a tack sensation to the user.

Furthermore, since commercially available sunscreen products often wash off in water, it is often necessary for sunscreen manufacturers to add additional levels of expensive sunscreen active ingredients to the compositions to ensure the desired high SPF is maintained even after immersion in water. For instance, current continuous spray products in the marketplace having a post-immersion SPF of at least 50 comprise anywhere from 25% to 50% active ingredients, with the remainder being alcohol. The inclusion of the additional sunscreen actives necessary to maintain a high SPF rating even after exposure to water can add to the cost of the sunscreen products.

It would thus be desirable to provide a sunscreen composition that has good water resistance properties, and also comprises a low level of sunscreen actives, as compared to currently available products, while maintaining a high SPF rating after immersion in water.

### SUMMARY OF THE DISCLOSURE

The present disclosure generally relates to sunscreen compositions. More particularly, the disclosure relates to a sunscreen composition having improved water resistance and a low overall percentage of sunscreen actives. To achieve the improved water resistance, a dimethicone derivative is incorporated into the sunscreen compositions.

In one aspect, the present disclosure is directed to a sunscreen composition comprising from about 38% (by total weight of the composition) to about 92% (by total weight of the composition) of a carrier, not more than about 25% (by total weight of the composition) of a sunscreen active, and from about 0.5% (by total weight of the composition) to about 5.0% (by total weight of the composition) of a dimethicone derivative selected from the group consisting of polyoxyethylene derivatized dimethicone, polyoxypropylene derivatized dimethicone, polyoxyethylene/polyoxypropylene derivatized dimethicone, and combinations thereof.

In another aspect, the present disclosure is directed to a sunscreen composition comprising a carrier, a sunscreen active, and a dimethicone derivative selected from the group consisting of polyoxyethylene derivatized dimethicone, polyoxypropylene derivatized dimethicone, polyoxyethylene/polyoxypropylene derivatized dimethicone, and combinations thereof, wherein from about 75% to about 100% of an SPF rating of the composition is retained following immersion in water for 80 minutes.

Other objects and features will be in part apparent and in part pointed out hereinafter.

### DETAILED DESCRIPTION OF THE DISCLOSURE

The present disclosure generally relates to sunscreen compositions. More particularly, the disclosure relates to sunscreen compositions having improved water resistance and a low overall percentage of sunscreen actives. To achieve the improved water resistance, a dimethicone derivative is incorporated into the sunscreen compositions.

For sunscreen products claiming to be very water resistant or waterproof, the advertised SPF rating must be determined following an 80 minute immersion in water, referred to herein as the post-immersion SPF. Typically, in order to produce a sunscreen composition having a desired post-immersion SPF rating, it is necessary to formulate the sunscreen composition with a static (i.e., pre-immersion) SPF significantly higher than the desired post-immersion SPF. This is done with the expectation that anywhere from about 25% to about 50% of the composition's SPF rating will be lost following the 80 minute water immersion. As a result, additional levels of expensive sunscreen actives must be added to achieve the needed high static SPF rating and desired post-immersion SPF.

It has now surprisingly been found that incorporating certain dimethicone derivatives into a sunscreen composition improves the water resistance of the composition and lowers the overall percentage of sunscreen actives needed to achieve a high SPF rating following water immersion.

In particular, the compositions of the present disclosure advantageously will retain from about 75% to about 100%, preferably from about 80% to about 100%, and more preferably from about 95% to about 100% of their SPF rating following 80 minutes of immersion in water, determined as described in the examples. Because there is little, if any, drop-off from the composition's static SPF to post-immersion SPF, the total amount of sunscreen actives needed to achieve the desired post-immersion SPF is lower than what would be required for compositions that do not comprise a dimethicone derivative as described herein. This effect occurs even in the absence of other waterproofing agents or additional film formers. Thus, it is not necessary to include additional waterproofing agents or film formers in the composition in order to achieve a sunscreen composition having good water resistance and a lower percentage of total sunscreen actives.

Without wishing to be bound to any particular theory, it is believed that when the sunscreen composition of the present disclosure is applied to skin, the dimethicone derivative creates a uniform film on the skin. As a result, sunscreen actives present in the composition are uniformly distributed throughout the film, and evenly deposited on the skin. The uniform distribution of actives on the skin improves the effectiveness of the sunscreen actives at absorbing light and also improves the adhesion of the actives to the skin. In a similar manner, if the sunscreen composition comprises a traditional waterproofing agent, the dimethicone derivative may improve the distribution and adherence of the waterproofing agent on the skin, resulting in a more water resistant film with higher retention on the skin than if the waterproofing agent were used alone.

The ability of the dimethicone derivatives used herein to improve the water resistance of sunscreen compositions is particularly unexpected, given the hydrophilic nature of these silicones. Most traditional waterproofing or film forming agents used to waterproof sunscreens have limited compatibility with water, and thus, as expected, are capable of forming a water resistant film on the skin. In contrast, the dimethicone derivatives used herein are more hydrophilic than traditional waterproofing agents. However, instead of washing off when immersed in water, as would be expected for compound having hydrophilic substituents, the dimethicone derivatives instead improve retention of sunscreen composition components on the skin. As previously mentioned, this is a distinct advantage when formulating very water resistant sunscreen compositions.

Thus, in certain aspects, the present disclosure is directed to sunscreen compositions comprising a carrier, a sunscreen active, and a dimethicone derivative. More particularly, the sunscreen compositions may comprise from about 38% (by total weight of the composition) to about 92% (by total weight of the composition) of a carrier, not more than about 25% (by total weight of the composition) of a sunscreen active, and from about 0.5% (by total weight of the composition) to about 5.0% (by total weight of the composition) of a dimethicone derivative. Preferably about 75% to about 100% of an SPF rating of the composition is retained following immersion in water for 80 minutes.

### Dimethicone Derivatives

Thus, the sunscreen compositions of the present disclosure advantageously comprise a dimethicone derivative. Preferably, the dimethicone derivatives used herein are selected from the group consisting of polyoxyethylene derivatized dimethicones, polyoxypropylene derivatized dimethicones, polyoxyethylene/polyoxypropylene derivatized dimethicones, and combinations thereof. In addition to improving the water resistance of the sunscreen compositions, the dimethicone derivatives have good inherent lubricity, and thus impart a pleasant skin feel to the sunscreen compositions.

As used herein, the term “polyoxyethylene derivatized dimethicone” is meant to include dimethicone polymers comprising a substituted or unsubstituted polyethylene glycol (PEG) functional group and methicone polymers comprising a substituted or unsubstituted PEG functional group. The polyoxyethylene derivatized dimethicone may be either pendant or linear. Pendant polyoxyethylene derivatized dimethicone has the following general structure:

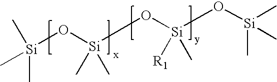

wherein R₁ is a substituted or unsubstituted polyethylene glycol functional group, x is any number from 0 to 350, and y is any number from 1 to 350. Polyoxyethylene derivatized dimethicones wherein x is 0 are methicone polymers comprising a substituted or unsubstituted PEG functional group.

Linear polyoxyethylene derivatized dimethicone has the following general structure:

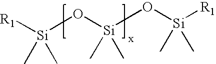

wherein R₁ is a substituted or unsubstituted polyethylene glycol functional group, and x is any number from 1 to 700.

Examples of preferred polyoxyethylene derivatized dimethicones suitable for use in the compositions of the present disclosure include Silsoft® dimethicones, available from Momentive (Wilton, Conn.), such as SF1488 (INCI designation: PEG-4 dimethicone), Silsoft® 805 (INCI designation: PEG-8 dimethicone; molecular weight: about 10,000); Silsoft® 810 (INCI designation: PEG-8 dimethicone; molecular weight: about 1,700), Silsoft® 840 (INCI designation: PEG-8 dimethicone; molecular weight: about 4,000), Silsoft® 870 (INCI designation: PEG-12 dimethicone; molecular weight: about 2,100), SF1288 (INCI designation: PEG-12 dimethicone); Silsoft® 875 (INCI designation: PEG-12 dimethicone); Silsoft® 880 (INCI designation: PEG-12 dimethicone; molecular weight: about 5,000); Silsoft® 895 (INCI designation: PEG-17 dimethicone; molecular weight: about 5,000), SF1388 (INCI designation: bis-PEG-20 dimethicone). The SF1488, Silsoft® 810, Silsoft® 870, and SF1388 are linear polyoxyethylene derivatized dimethicones, while the Silsoft® 805, Silsoft® 840, SF1288, Silsoft® 875, Silsoft® 880, and Silsoft® 895 are pendant polyoxyethylene derivatized dimethicones.

Other examples of suitable polyoxyethylene derivatized dimethicones include PEG-1 dimethicone, PEG-3 dimethicone, PEG-6 dimethicone, PEG-7 dimethicone, PEG-9 dimethicone, PEG-10 dimethicone, PEG-14 dimethicone, and the like.

The polyoxyethylene derivatized dimethicone may also include PEG derivatized dimethicones that have additional moieties added to the polymer, including bis-PEG-15 methyl ether dimethicone, dimethicone PEG-15 acetate, dimethicone PEG-8 adipate, dimethicone PEG-7 avocadoate, dimethicone PEG-8 avocadoate, dimethicone PEG-8 beeswax, dimethicone PEG-8 benzoate, dimethicone PEG-8 borageate, dimethicone PEG-7 cocoate, dimethicone PEG-7 isostearate, dimethicone PEG-8 isostearate, dimethicone PEG-7 lactate, dimethicone PEG-8 lanolate, dimethicone PEG-8 laurate, dimethicone PEG-8 meadowfoamate, dimethicone PEG-7 octyldodecyl citrate, dimethicone PEG-7 olivate, dimethicone PEG-8 olivate, dimethicone PEG-7 phosphate, dimethicone PEG-8 phosphate, dimethicone PEG-10 phosphate, dimethicone PEG-7 phthalate, dimethicone PEG-8 phthalate, dimethicone PEG-8 polyacrylate, dimethicone PEG-7 succinate, dimethicone PEG-8 succinate, dimethicone PEG-7 sulfate, dimethicone PEG-7 undecylenate, lauryl dimethicone PEG-10 phosphate, lauryl PEG-8 dimethicone, lauryl PEG-10 methyl ether dimethicone, lauryl PEG-9 polydimethylsiloxyethyl dimethicone, PEG-6 methyl ether dimethicone, PEG-7 methyl ether dimethicone, PEG-8 methyl ether dimethicone, PEG-9 methyl ether dimethicone, PEG-10 methyl ether dimethicone, PEG-11 methyl ether dimethicone, PEG-32 methyl ether dimethicone, PEG-12 methyl ether lauroxy PEG-5 amidopropyl dimethicone, perfluorononylethyl carboxydecyl PEG-8 dimethicone, perfluorononylethyl carboxydecyl PEG-10 dimethicone, perfluorononylethyl PEG-8 dimethicone, and combinations thereof.

Preferred polyoxyethylene derivatized dimethicones are selected from the group consisting of PEG-3 dimethicone, PEG-4 dimethicone, PEG-8 dimethicone, PEG-12 dimethicone, PEG-17 dimethicone, bis-PEG-20 dimethicone, and combinations thereof.

The dimethicone derivative may also be a polyoxypropylene derivatized dimethicone. As used herein, the term “polyoxypropylene derivatized dimethicone” is meant to include dimethicone polymers comprising a substituted or unsubstituted polypropylene glycol (PPG) functional group and methicone polymers comprising a substituted or unsubstituted PPG functional group. Like discussed above with regard to the polyoxyethylene derivatized dimethicones, the polyoxypropylene derivatized dimethicone may be either pendant or linear. Pendant and linear polyoxypropylene derivatized dimethicones have the same general structures as set forth above for pendant and linear polyoxyethylene derivatized dimethicones, respectively, except R₁ is a substituted or unsubstituted polypropylene glycol functional group.

Examples of preferred polyoxypropylene derivatized dimethicones suitable for use in the compositions of the present disclosure include Silsoft® dimethicones, available from Momentive (Wilton, Conn.), such as Silsoft® 900 (INCI designation: PPG-12 dimethicone; molecular weight: about 2,800), Silsoft® 910 (INCI designation: PPG-12 dimethicone; molecular weight: about 13,000), and Silsoft® 920 (INCI designation: PPG-12 dimethicone). Silsoft® 900 is a linear polyoxypropylene derivatized dimethicone, while Silsoft® 910 and Silsoft® 920 are a pendant polyoxypropylene derivatized dimethicones.

Other examples of suitable polyoxypropylene derivatized dimethicones include PPG-2 dimethicone, PPG-27 dimethicone, and the like. The polyoxypropylene derivatized dimethicone may also include PPG derivatized dimethicones that have additional moieties added to the polymer, including PPG-12 butyl ether dimethicone, and the like.

Thus, in one embodiment, the dimethicone derivative is a polyoxypropylene derivatized dimethicone selected from the group consisting of PPG-12 dimethicone, PPG-2 dimethicone, PPG-27 dimethicone, PPG-12 butyl ether dimethicone, and combinations thereof.

The dimethicone derivative may also be a polyoxyethylene/polyoxypropylene derivatized dimethicone. As used herein, the term “polyoxyethylene/polyoxypropylene derivatized dimethicone” is meant to include dimethicone polymers comprising a substituted or unsubstituted polyoxyethylene/polyoxypropylene (PEG/PPG) functional group and methicone polymers comprising a substituted or unsubstituted PEG/PPG functional group. Like discussed above with regard to the polyoxyethylene derivatized dimethicones and the polyoxypropylene derivatized dimethicones, the polyoxyethylene/polyoxypropylene derivatized dimethicone may be either pendant or linear. Pendant and linear polyoxyethylene/polyoxypropylene derivatized dimethicones have the same general structures as set forth above for pendant and linear polyoxyethylene derivatized dimethicones, respectively, except R₁ is a substituted or unsubstituted polyethylene glycol/polypropylene glycol functional group.

Examples of preferred polyoxyethylene/polyoxypropylene derivatized dimethicones suitable for use in the compositions of the present disclosure include Silsoft® dimethicones, available from Momentive (Wilton, Conn.), such as Silsoft® 430 (INCI designation: PEG-20/PPG-23 dimethicone; molecular weight: about 29,000), SF1188A (INCI designation: PEG/PPG 20-15 dimethicone), Silsoft® 440 (INCI designation: PEG-20/PPG-23 dimethicone; molecular weight: about 20,000), and Silsoft® 475 (INCI designation: PEG-23/PPG-6 dimethicone; molecular weight: about 19,000). Silsoft® 430, SF1188A, Silsoft® 440, and Silsoft® 475 are all pendant polyoxyethylene/polyoxypropylene derivatized dimethicones.

Other examples of suitable polyoxyethylene/polyoxypropylene derivatized dimethicones include PEG-3/PPG-10 dimethicone, PEG-4/PPG-12 dimethicone, PEG-6/PPG-11 dimethicone, PEG-8/PPG-14 dimethicone, PEG-8/PPG-26 dimethicone, PEG-10/PPG-2 dimethicone, PEG-12/PPG-16 dimethicone, PEG-12/PPG-18 dimethicone, PEG-14/PPG-4 dimethicone, PEG-15/PPG-15 dimethicone, PEG-16/PPG-2 dimethicone, PEG-16/PPG-8 dimethicone, PEG-17/PPG-18 dimethicone, PEG-18/PPG-6 dimethicone, PEG-18/PPG-18 dimethicone, PEG-19/PPG-19 dimethicone, PEG-20/PPG-6 dimethicone, PEG-20/PPG-15 dimethicone, PEG-20/PPG-20 dimethicone, PEG-20/PPG-29 dimethicone, PEG-22/PPG-23 dimethicone, PEG-22/PPG-24 dimethicone, PEG-23/PPG-6 dimethicone, PEG-25/PPG-25 dimethicone, PEG-27/PPG-27 dimethicone, PEG-30/PPG-10 dimethicone, and PPG-4-oleth-10 dimethicone (i.e., PEG-10/PPG-4 dimethicone).

The polyoxyethylene/polyoxypropylene derivatized dimethicone may also include PEG/PPG derivatized dimethicones that have additional moieties added to the polymer, including Bis-PEG-16/PPG-16 PEG-16/PPG-16 dimethicone, dimethicone PEG-20/PPG-23 benzoate, dimethicone PEG-7/PPG-4 phosphate, dimethicone PEG-12/PPG-4 phosphate, PEG-28/PPG-21 acetate dimethicone, PEG/PPG-20/22 butyl ether dimethicone, PEG/PPG-22/22 butyl ether dimethicone, PEG/PPG-23/23 butyl ether dimethicone, PEG-24/PPG-18 butyl ether dimethicone PEG-27/PPG-9 butyl ether dimethicone PEG-24/PPG-24 methyl ether glycidoxy dimethicone, PEG-10/PPG-3 oleyl ether dimethicone, and the like.

Preferred polyoxyethylene/polyoxypropylene derivatized dimethicones are selected from the group consisting of PEG-20/PPG-23 dimethicone, PEG/PPG 20-15 dimethicone, PEG-23/PPG-6 dimethicone, and combinations thereof. In one preferred embodiment, the dimethicone derivative is a PEG-20/PPG-23 dimethicone, such as Silsoft® 440.

As noted above, the polyoxyethylene/polyoxypropylene derivatized dimethicones may be methicone polymers comprising a PEG/PPG functional group.

Suitable examples of such polymers are the Silsoft® methicones, available from Momentive (Wilton, Conn.), such as Silsoft® 305 (INCI designation: PEG-5/PPG-3 methicone; molecular weight 600).

Typically, the sunscreen compositions comprise dimethicone derivatives in an amount of from about 0.5% (by total weight of the composition) to about 5.0% (by total weight of the composition), more preferably, from about 1.0% (by total weight of the composition) to about 5.0% (by total weight of the composition), and still more preferably in an amount of from about 2.0% (by total weight of the composition) to about 4.0% (by total weight of the composition).

### Sunscreen Actives

As noted above, the sunscreen compositions of the present disclosure also comprise at least one sunscreen active. The sunscreen active may be organic or inorganic, or a combination of both may be used. Suitable sunscreen actives used in the present disclosure include UV absorbers or blockers. UV absorbers may be a UVB or UVA absorber. In some embodiments, the sunscreen active includes more than one organic sunscreen active and at least one inorganic sunscreen active. In some embodiments, the sunscreen active includes only a physical blocker sunscreen.

Any sunscreen active known in the art may be used in the compositions described herein. The term “sunscreen active” is intended to include any ultraviolet ray-blocking compounds exhibiting absorption or blockage within the wavelength region between about 290 and 420 nm, or infrared radiation. Sunscreen actives are typically classified into five groups based upon their chemical structure: amino benzoates; salicylates; cinnamates; benzophenones, and miscellaneous chemicals including menthyl anthralinate and digalloyl trioleate. Inorganic sunscreens may also be used including titanium dioxide, zinc oxide, iron oxide, and polymer particles such as those of polyethylene and polyamides.

Examples of suitable aminobenzoic acids include aminobenzoic acid, its salts, and its derivatives, such as ethyl, isobutyl, and glyceryl esters, p-dimethylaminobenzoic acid, and 4-aminobenzoic acid derivatives, including 4-(dimethylamino)-benzoic acid-2-ethylhexyl ester, 4-(dimethylamino)-benzoic acid-2-octyl ester and 4-(dimethylamino)-benzoic acid amyl ester, and the like.

Examples of suitable cinnamates include cinnamic acid derivatives such as methyl and benzyl esters, α-phenyl cinnamonitrile, butyl cinnamoyl pyruvate, and the like.

Examples of cinnamic acid esters include octinoxate (octyl methoxycinnamate), 4-methoxycinnamic acid propyl ester, 4-methoxycinnamic acid isoamyl ester, and octocrylene (2-ethylhexyl-2-cyano-3,3-diphenylacrylate). Other suitable sunscreen actives include dihydroxycinnamic acid derivatives, trihydroxycinnamic acid derivatives, and the like.

Examples of suitable salicylates include amyl, phenyl, benzyl, menthyl, glyceryl, and dipropylene glycol esters. Specific examples of salicylic acid esters include octisalate (2-ethylhexyl salicylate), salicylic acid-4-isopropylbenzyl ester, and salicylic acid homomethyl ester.

Examples of suitable benzophenones include oxybenzone, sulisobenzone, dioxybenzone, benzoresorcinol, 2,2′,4,4′-tetrahydroxybenzophenone, 2,2′-dihydroxy 4,4′-dimethoxybenzophenone, octabenzone, and the like. Suitable derivatives of benzophenone include, for example, 2-hydroxy-4-methoxybenzo-phenone, 2-hydroxy-4-methoxy-4′-methylbenzophenone, 2,2′-dihydroxy-4-methoxy-benzophenone, and the like. Other benzophenone derivatives include sulfonic acid derivatives of benzophenones, preferably 2-hydroxy-4-methoxybenzophenone-5-sulfonic acid and salts thereof.

Other suitable sunscreen actives include, for example, 3-benzylidene camphor or 3-benzylidene norcamphor and derivatives thereof, such as 3-(4-methylbenzylidene)-camphor; esters of benzalmalonic acid, such as 4-methoxybenzalmalonic acid di-2-ethylhexyl ester; triazine derivatives such as, for example, 2,4,6-trianilino-(p-carbo-2′-ethyl-1′-hexyloxy)-1,3,5-triazine, and Octyl Triazone; avobenzone (butyl methoxydibenzoylmethane); ketotricyclo(5.2.1.0)decane derivatives; 2-phenylbenzimidazole-5-sulfonic acid and alkali metal, alkaline earth metal, ammonium, alkylammonium, alkanolammonium and glucammonium salts thereof; sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert-butylphenyl)-3-(4′-methoxyphenyl)-propane-1,3-dione, 4-tert-butyl-4′-methoxydibenzoyl methane (Parsol 1789) or 1-phenyl-3-(4′-isopropylphenyl)-propane-1,3-dione. The UV-A and UV-B filters may of course also be used in the form of mixtures.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Aeroxide TiO₂ T805 (Degussa) and Eusolex T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used.

Besides the primary sun protection factors mentioned above, secondary sun protection factors of the antioxidant type may also be used. Secondary sun protection factors of the antioxidant type interrupt the photochemical reaction chain which is initiated when UV rays penetrate into the skin. Secondary sun protection factors of the antioxidant type are particularly useful in emulsion-based sunscreen compositions. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μ mole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxide-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

In preferred embodiments, the sunscreen actives are FDA approved or approved for use in the European Union. Examples of suitable FDA approved sunscreen actives are described in the Final Over-the-Counter Drug Products Monograph on Sunscreens (Federal Register, 1999:64:27666-27693), herein incorporated by reference. It should be understood that the specific sunscreen actives and amounts thereof that are approved for use in the United States or European Union are subject to periodic change. As such, the specific examples and amounts set forth herein are not intended to be limiting.

For example, for a product marketed in the United States, preferred cosmetically-acceptable sunscreen actives and concentrations (reported as a percentage by total weight of the composition) may include aminobenzoic acid (i.e., para-aminobenzoic acid or PABA) in amounts up to 15%; avobenzone (i.e., butyl methoxy dibenzoylmethane) in amounts of up to 3.0%; cinoxate (2-ethoxyethyl p-methoxycinnamate) in amounts up to 3.0%; dioxybenzone (i.e., benzophenone-8) in amounts up to 3.0%; homosalate in amounts up to 15.0%; menthyl anthranilate in amounts up to 5.0%; octocrylene (i.e., 2-ethylhexyl-2-cyano-3,3 diphenylacrylate) in amounts up to 10%; octinoxate (i.e., octyl methoxycinnamate) in amounts up to 7.5%; octisalate (i.e., octyl salicylate or 2-ethylhexyl salicylate) in amounts up to 5.0%; oxybenzone (i.e., benzophenone-3) in amounts up to 6.0%; padimate O (i.e., octyl dimethyl PABA) in amounts up to 8.0%; phenylbenzimidazole sulfonic acid in amounts up to 4.0%; sulisobenzone in amounts up to 10%; titanium dioxide in amounts up to 25.0%; trolamine salicylate in amounts up to 12.0%; and zinc oxide in amounts up to 25.0%. Combinations of these actives may also be used.

For a product marketed in the European Union, preferred sunscreen actives and concentrations (reported as a percentage by total weight of the composition) may include: PABA in amounts up to 5.0%, camphor benzalkonium methosulfate in amounts up to 6.0%, homosalate in amounts up to 10.0%, oxybenzone in amounts up to 10.0%, phenylbenzimidazole sulfonic acid in amounts up to 8.0%, terephthalidene dicamphor sulfonic acid in amounts up to 10.0%, avobenzone in amounts up to 5.0%, benzylidene camphor sulfonic acid in amounts up to 6.0%, octocrylene in amounts up to 10.0%, polyacrylamidomethyl benzylidene camphor in amounts up to 6.0%, octinoxate in amounts up to 10.0%, PEG-25 PABA in amounts up to 10.0%, isoamyl p-methoxycinnamate in amounts up to 10.0%, ethylhexyl triazone in amounts up to 5.0%, drometrizole trielloxane in amounts up to 15.0%, diethylhexyl butamido triazone in amounts up to 10.0%, 4-methylbenzylidene camphor in amounts up to 4.0%, 3-benzylidene camphor in amounts up to 2.0%, octisalate in amounts up to 5.0%, ethylhexyl dimethyl PABA in amounts up to 8.0%, sulisobenzone in amounts up to 5.0%, methylene bis-benztriazolyl tetramethylbutylphenol in amounts up to 10.0%, disodium phenyl dibenzimidazole tetrasulfonate in amounts up to 10.0%, bis-ethylhexyloxyphenol methoxyphenol triazine in amounts up to 10.0%, methylene bisbenzotriazolyl tetramethylbutylphenol in amounts up to 10.0% (also called TINOSORB® M), and bisethylhexyloxyphenol methoxyphenyl triazine in amounts up to 10.0% (also called TINOSORB® S). Combinations of these actives may also be used.

In a preferred embodiment, the sunscreen active is selected from the group consisting of aminobenzoic acid, avobenzone, cinoxate, dioxybenzone, homosalate, menthyl anthranilate, octocrylene, octinoxate, octisalate, oxybenzone, padimate O, phenylbenzimidazole sulfonic acid, sulisobenzone, titanium dioxide, trolamine salicylate, zinc oxide, camphor benzalkonium methosulfate, terephthalidene dicamphor sulfonic acid, benzylidene camphor sulfonic acid, polyacrylamidomethyl benzylidene camphor, PEG-25 PABA, isoamyl p-methoxycinnamate, ethylhexyl triazone, drometrizole trielloxane, diethylhexyl butamido triazone, 4-methylbenzylidene camphor, 3-benzylidene camphor; ethylhexyl dimethyl PABA, methylene bis-benztriazolyl tetramethylbutylphenol, disodium phenyl dibenzimidazole tetrasulfonate, bis-ethylhexyloxyphenol methoxyphenol triazine, methylene bisbenzotriazolyl tetramethylbutylphenol, bisethylhexyloxyphenol methoxyphenyl triazine, and combinations thereof.

In one preferred embodiment, the sunscreen active is selected from the group consisting of avobenzone, octinoxate, oxybenzone, octisalate, octocrylene, homosalate, and combinations thereof. In a particular embodiment, the sunscreen active comprises about 1.0% (by total weight of the composition) of avobenzone, about 7.5% (by total weight of the composition) of octinoxate, about 5.0% (by total weight of the composition) of oxybenzone, about 5.0% (by total weight of the composition) of octisalate, about 0.8% (by total weight of the composition) of octocrylene, and about 4.0% (by total weight of the composition) of homosalate.

Other suitable sunscreen actives include UV absorbers sold under the name TINOSORB® UV absorber, available from Ciba, or EUSOLEX® UV filters, such as EUSOLEX® 232 (INCI designation: phenylbenzimidazole sulfonic acid), EUSOLEX® 4360 (INCI designation: benzophenone-3), EUSOLEX® 6007 (INCI designation: ethylhexyl dimethyl PABA), EUSOLEX® 6300 (INCI designation: 4-methylbenzylidene camphor), EUSOLEX® 9020 (INCI designation: butyl methoxydibenzoyl methane), EUSOLEX® HMS (INCI designation: homosalate), EUSOLEX® OCR (INCI designation: octocrylene), EUSOLEX® OS (INCI designation: ethylhexyl salicylate), EUSOLEX® 2292 (INCI designation: ethylhexyl methoxycinnamate, BHT), EUSOLEX® T (INCI designation: titanium dioxide, water (aqua), simethicone), EUSOLEX® T-AVO (INCI designation: titanium dioxide, silica), EUSOLEX® T-45D, EUSOLEX® T-Oleo (INCI designation: butylene glycol dicaprylate/dicaprate, titanium dioxide, silica, polyglyceryl-2-dipolyhydroxystearate), EUSOLEX® T-S (INCI designation: titanium dioxide, alumina, stearic acid), and EUSOLEX® T-2000 (INCI designation: titanium dioxide, alumina, simethicone), available from Merck.

It will be appreciated by those of skill in the art that the various sunscreen actives may be added to the composition all at once, or in groups, or separately. In some embodiments, the sunscreen active comprises at least two components.

As noted above, inclusion of a dimethicone derivative, as described herein, in the sunscreen compositions of the present disclosure advantageously improves the water resistance of the composition and lowers the overall percentage of sunscreen actives needed to achieve a high post-immersion SPF rating.

The total amount of sunscreen active present in the compositions may vary depending on the target post-immersion SPF of the composition. Typically, the sunscreen compositions of the present disclosure will have a static SPF of from about 15 to about 90, and more typically from about 30 to about 60, and a post-immersion SPF of at least about 25, more preferably at least about 40, and more preferably at least about 50. As such, the compositions will preferably comprise a total amount of sunscreen active sufficient to achieve these target SPF ratings.

Preferably, however, the composition will comprise not more than about 25% (by total weight of the composition) of sunscreen actives. Inclusion of sunscreen actives into the compositions in amounts of about 25% (by total weight of the composition) will preferably achieve a static SPF of at least about 50, and a post-immersion SPF of at least about 50.

In other embodiments, the composition will comprise not more than about 20% (by total weight of the composition) of sunscreen actives. Inclusion of sunscreen actives into the compositions in amounts of about 20% (by total weight of the composition) will preferably achieve a static SPF of at least about 40, and more typically, at least about 50, and a post-immersion SPF of at least about 40.

Thus, in particular embodiments, when the composition has a post-immersion SPF rating of at least about 50, the composition preferably will comprise not more than about 25% (by total weight of the composition) of sunscreen actives, and when the composition has a post-immersion SPF rating of at least about 40, the composition preferably will comprise not more than about 20% (by total weight of the composition) of sunscreen actives.

It is to be understood that static and post-immersion SPF ratings below 40 may also be achieved by lowering the amount of sunscreen actives included in the composition.

Stated another way, the sunscreen compositions of the present disclosure preferably have a ratio of static SPF to total actives concentration of from about 1 to about 8, and preferably from about 1.8 to about 2.4. The sunscreen compositions of the present disclosure will also preferably have a ratio of post-immersion SPF to total actives concentration of from about 1 to about 6, and preferably from about 1.8 to about 2.4.

### Carriers

The sunscreen compositions of the present disclosure further comprise a carrier. The sunscreen compositions are preferably formulated as a liquid suitable for spray on application. Upon application of the composition to the skin, the carrier evaporates and leaves behind a thin film comprising the active ingredients, the dimethicone derivative, and any other composition components, deposited on the skin. The film, containing the sunscreen actives, remains on the skin and protects the skin from ultraviolet radiation and damage resulting therefrom.

Since the sunscreen compositions are preferably formulated for spray on application, it is generally preferable for the carrier to be capable of evaporating upon application of the composition to the skin. Examples of suitable carriers include alcohols, water, volatile solvents such as ether solvents, and combinations thereof.

Examples of suitable volatile ether solvents include dimethyl ether, diethyl ether, and combinations thereof. Examples of suitable commercially available volatile ether solvents include Cosmetic Fluid CF-76 (INCI designation: ethyl perfluorobutyl ether/ethyl perfluoroisobutyl ether) and Cosmetic Fluid CF-61 (INCI designation: methyl perfluorobutyl ether/methyl perfluoroisobutyl ether), both available from 3M.

In a preferred embodiment, the carrier is an alcohol. The alcohol may be a glycol, such as ethylene glycol, propylene glycol, butylene glycol, pentylene glycol, hexylene glycol, dipropylene glycol, ethoxydiglycol, and the like. Other examples of suitable alcohols include methyl alcohol, ethyl alcohol, propyl alcohol, isopropyl alcohol, butyl alcohol, and the like. Preferably, the alcohol is a specially denatured alcohol.

The amount of carrier present in the composition will vary depending on the amount of sunscreen actives, dimethicone derivatives, and other components, but typically is present in an amount of from about 38% (by total weight of the composition) to about 92% (by total weight of the composition), and more typically from about 59.7% (by total weight of the composition) to about 73.7% (by total weight of the composition).

### Optional Components

Optionally, the sunscreen compositions of the present disclosure may further comprise a component that provides a film barrier, typically a hydrophobic layer that serves to maintain the sunscreen actives on the skin after immersion in water, such as a waterproofing agent or other film former. As noted above, it is believed the dimethicone derivatives of the present disclosure act to improve the effectiveness of waterproofing agents or film formers in the sunscreen composition, in a manner similar to that described above for sunscreen actives.

Preferably, the waterproofing agents used herein are acrylic co-polymers. A particularly preferred waterproofing agent is DERMACRYL® 79 (INCI designation: Acrylates/Octylacrylamide copolymer), marketed by National Starch and Chemical, which is a copolymer between N-octylacrylamide and either acrylic acid, methacrylic acid, or simple esters thereof. Another exemplary liquid acrylic copolymer formulation is DERMACRYLOAQF (INCI designation: acrylates copolymer), marketed by National Starch and Chemical. Other examples of suitable acrylic copolymers include acrylates/C₁₂₋₂₂ alkyl methacrylate copolymer, Ganex P904 (INCI designation: butylated PVP) (available from ISP), Advantage Plus (INCI designation: VA/butyl maleate/isobornyl acrylate copolymer) (available from ISP), and Advantage HC-37 (INCI designation: vinyl caprolactam/VP/dimethylaminoethyl methacrylate copolymer) (available from ISP), and the like. Acrylic co-polymers may be included in the sunscreen compositions in amounts of from about 0.5% (by total weight of the composition) to about 5.0% (by total weight of the composition), preferably from about 1.0% (by total weight of the composition) to about 5.0% (by total weight of the composition), and preferably from about 2.0% (by total weight of the composition) to about 4.0% (by total weight of the composition).

Other suitable waterproofing agents and film formers are well-known in the art and include, without limitation, petrolatum, emollient esters, lanolin derivatives (e.g., acetylated lanolins), superfatted oils, cyclomethicone, cyclopentasiloxane, dimethicone, natural and synthetic oils, fatty acids, fatty alcohols, waxes, and the like.

In addition, many emollients may also exhibit a film forming function in that they provide a water-resistant barrier on the skin. Thus, the sunscreen compositions of the present disclosure may optionally include an emollient. A particularly preferred emollient is butyloctyl salicylate, sold under the name HallBrite® BHB, available from HallStar®. Emollients such as butyloctyl salicylate may be included in the composition in amounts preferably not more than about 10.0% (by total weight of the composition), more preferably in amounts of from about 0.1% (by total weight of the composition) to about 10.0% (by total weight of the composition), preferably from about 1.0% (by total weight of the composition) to about 8.0% (by total weight of the composition), and more preferably about 3.5% (by total weight of the composition) to about 7.0% (by total weight of the composition).

Other emollients suitable for inclusion in the sunscreen compositions of the present disclosure may include water-insoluble emollients that include fatty acids such as oleic and stearic; fatty alcohols such as cetyl, and hexadecyl; esters such as diisopropyl adipate, benzoic acid esters of C₁-C₁₅ alcohols, and isononyl isononanoate; alkanes such as mineral oil; ethers such as polyoxypropylene butyl ethers and polyoxypropylene cetyl ethers; and natural and synthetic oils.

A preservative, such as an antimicrobial preservative, can also be optionally added to the sunscreen compositions as described herein. An antimicrobial preservative is a compound or substance that destroys, prevents, or inhibits the multiplication/growth of microorganisms in the sunscreen composition and may offer some protection from oxidation of the sunscreen composition. Suitable preservatives include, for example, the lower alkyl esters of para-hydroxybenzoates such as methylparaben, propylparaben, isobutylparaben, and mixtures thereof, benzyl alcohol, DMDM Hydantoin, and benzoic acid.

Another optional ingredient that can be added to the sunscreen compositions described herein is antioxidants. An antioxidant is a natural or synthetic substance added to the sunscreen composition to protect it from decay or deterioration due to the reaction with oxygen in the air. Antioxidants prevent deterioration which may lead to the generation of rancidity and non-enzymatic browning reaction products. Suitable antioxidants include, for example, propyl, octyl and dodecyl esters of gallic acid, butylated hydroxyanisole, butylated hydroxytoluene, nordihydroguaiaretic acid, vitamin E, vitamin E acetate, vitamin C, lipoic acid, ubiquinone, Apple Green Tea, Arnica Special, Avocado GW, Bell Pepper Fruit, Black Currant B, Black Currant Green Tea, Blueberry Fruit, Cabbage Rose Extract, Camellia sinensis, Canadian Willowherb, Carrot Root, Camellia Oleifera Extract, Common thyme, Cranberry Green Tea, Echinacea Dry Aqueous Extract, Fennel Fruit, Ginkgo Biloba, Glycine Max (soybean seed), Goldenseal, Grapefruit, Grape Seed Extract and constituents thereof (proanthocyanidins), Grapefruit Green Tea, Green Tea, catechin constituents of Green Tea that include epigallocatechin gallate, epicathechin gallate, Green Tea Extra, Green Tea HS, Lime Blossom, Orange Green Tea, Rosemary Plant, Sea Parsley, St. John's Wort W/S, Strawberry Fruit, Tomato Root, Turkish Oregano, Wheat Seed, White Mistle Toe, White Tea, Yarrow, Yucca 70, Yucca Extract Powder, and combinations thereof.

Fragrances can also be added to the sunscreen compositions described herein. Fragrances are aromatic compounds which can impart an aesthetically pleasing aroma to the sunscreen composition. Typical fragrances include, for example, aromatic materials extracted from botanical sources such as rose petals, gardenia blossoms, and jasmine flowers which can be used alone or in any combination to create essential oils. Additionally, alcoholic extracts may be prepared for fragrances. The fragrance may be included in the composition in amounts of from about 0.1% (by total weight of the composition) to about 2.0% (by total weight of the composition), and preferably from about 0.5% (by total weight of the composition) to about 1.0% (by total weight of the composition).

Another ingredient that may optionally be added to the sunscreen composition in some embodiments of the present invention is humectants. A humectant is a moistening agent that promotes retention of water due to its hydroscopic properties, and moisturizes the skin. One or more humectants may be introduced into the sunscreen composition in an amount of from about 0.1% (by total weight of the composition) to about 10.0% (by total weight of the composition).

Suitable humectants for inclusion in the sunscreen compositions described herein include, for example, glycerin, sorbitol, polyols, sugars, hydrogenated starch hydrolysates, salts of PCA, lactates, and urea. A particularly preferred humectant is glycerin.

The sunscreen compositions may also optionally comprise a rheology modifier. Suitable rheology modifiers include combinations of alpha-olefins and styrene or polyethylene alone or in combination with mineral oil or petrolatum, di-functional alpha-olefins and styrene alone or in combination with mineral oil or petrolatum, combinations of alpha olefins and isobutene, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene copolymers, styrene-ethylene/propylene copolymers, silicas, cellulosics, gums, starches, modified starches, acrylate based rheology modifiers such as acrylates/C10-30 alkyl acrylate crosspolymer, silica, and silicate derivatives such as magnesium aluminum silicate. The compositions may comprise from about 0.1% (by total weight of the composition) to about 5.0% (by total weight of the composition) of rheology modifiers.

Another ingredient that may optionally be added to the sunscreen compositions described herein is light scattering or reflecting agents, such as silica, fumed silica, SunSpheres® powder (available from Rohm and Haas), aluminum oxide, and combinations thereof. The light scattering or reflecting agents may be included in the compositions in amounts of from about 0.01% (by total weight of the composition) to about 5.0% (by total weight of the composition), and more typically from about 0.05% (by total weight of the composition) to about 3.0% (by total weight of the composition).

The sunscreen compositions described herein may also optionally comprise aesthetic modifiers, such as DRYFLO® Elite BN (INCI designation: aluminum starch octenyl succinate/boron nitride), starches and cellulosics, Nylon 12, and the like. The aesthetic modifiers may be included in the compositions in amounts of from about 0.01% (by total weight of the composition) to about 5.0% (by total weight of the composition).

Additional optional ingredients which can be incorporated into the sunscreen compositions include vitamins, skin protectants, powders, skin conditioners, botanicals, natural extracts, alpha hydroxy acids, moisturizers, fats and oils, lipids, fatty alcohols, fatty acids, colorants, pigments, optical brighteners, and essential oils.

As described above, the sunscreen compositions of the present disclosure are preferably formulated into liquid compositions for spray-on application. In certain embodiments, however, the sunscreen compositions may be formulated into a variety of other sunscreen products including, for example, substantially water-free oils, creams, gels, lotions, aqueous/alcoholic solutions, emulsions such as O/W or W/O emulsions, sprayable emulsions, stick preparations and the like. Depending on the particular application, the compositions may thus contain a number of other auxiliaries and additives that may serve in combination with the carriers described herein, to form a base for the sunscreen product. Examples of suitable additives include for example, oil components, emulsifiers, surfactants, pearlizing waxes, consistency factors, thickeners, superfatting agents, stabilizers, polymers, silicone compounds, fats, waxes, lecithins, phospholipids, and the like.

In one preferred embodiment, the sunscreen composition comprises at least one sunscreen active, a carrier, a dimethicone derivative, as described herein, a waterproofing agent, an emollient, and fragrance. Preferably, the sunscreen active comprises avobenzone, octinoxate, oxybenzone, octisalate, octocrylene, and homosalate; the dimethicone derivative is a PEG-20/PPG-23 dimethicone; the waterproofing agent is an acrylates/octylacrylamide copolymer; the emollient is butyloctyl salicylate; and the carrier is an alcohol.

Having described the disclosure in detail, it will be apparent that modifications and variations are possible without departing from the scope of the disclosure defined in the appended claims.

### EXAMPLES

The following non-limiting examples are provided to further illustrate the present disclosure.

### Test Methods

### Sample Preparation and Static SPF Measurements

All SPF measurements were made in vitro using a UV-1000S Ultraviolet Transmittance Analyzer, available from Lapsphere, and VITRO-SKIN® synthetic skin substitute, available from IMS, Inc. (Orange, Conn.). To begin, the VITRO-SKIN® synthetic skin was hydrated for 24 hours in a controlled humidity chamber per the protocol suggested by the manufacturer. For each sample, a 1″×2″ section of VITRO-SKIN® was cut. The VITRO-SKIN® was placed rough side up on a foam block used to simulate the flexibility of human flesh, and a small micropipetter was used to dispense 100 μl of the sunscreen composition sample over the center area of the VITRO-SKIN® substrate. The sunscreen composition was gently rubbed in with a gloved finger for 20 to 30 seconds, mimicking the application of sunscreen to a body. Once the sunscreen was spread, the sample was transferred to a 6 cm×6 cm glassless slide mount and allowed to dry for 15 minutes. SPF measurements were made using the UV-1000S Ultraviolet Transmittance Analyzer. The SPF values given herein are the average of five measurements taken at different locations on each sample.

### Water Immersion Test and Post-Immersion SPF

The following test was used to determine the percent of an SPF rating for the sunscreen compositions that was retained following immersion in water for 80 minutes. When the terms “post-immersion SPF” and “static SPF” are used herein, the post-immersion SPF refers to the SPF of the sunscreen composition after being subjected to the following water immersion test, and the “static SPF” refers to the SPF of the sunscreen composition prior to immersion in water for 80 minutes, as described herein. All SPF values and values for percent SPF rating retained following water immersion given herein are based on in vitro testing.

To begin, the sunscreen samples were applied to hydrated VITRO-SKIN® synthetic skin substrate and the static SPF of the composition was measured as described above. The sunscreen composition-treated VITRO-SKIN® samples were then transferred to a water bath at 40° C. with agitation at 300 rpm for 80 minutes. The samples were removed from the water bath after 80 minutes, patted dry, and the post-immersion SPF of the samples was tested using the Labsphere UV-1000S Ultraviolet Transmittance Analyzer. The post-immersion SPF values given herein are the average of five measurements taken at different locations on each sample. The percent SPF rating retained following immersion in water for 80 minutes was determined by dividing the post-immersion SPF rating by the static SPF rating.

### Example 1

In this example, a sunscreen composition comprising sunscreen actives, Silsoft® 440, a carrier, a waterproofing agent, an emollient, and a fragrance was prepared and the static SPF, post immersion SPF, and % SPF retained following the water immersion test was determined according to the procedures described in the Test Methods section. The following components were used to prepare the composition:

TABLE 1

| | | |
|---|---|---|
| Trade Name | INCI Name | % weight |

Phase A

| | | |
|---|---|---|
| SD Alcohol-200 Proof | SD Alcohol 40B | 72.70 |
| Dermacryl ® 79 | Acrylates/octylacrylamide | 2.00 |
| | copolymer | |

Phase B

| | | |
|---|---|---|
| Escalol ® 557 | Octinoxate | 7.50 |
| Escalol ® 587 | Octisalate | 5.00 |
| Escalol ® 597 | Octocrylene | 0.80 |
| Escalol ® 517 | Avobenzone | 1.00 |
| Escalol ® 567 | Oxybenzone | 5.00 |
| Silsoft ® 440 | PEG-20/PPG-23 dimethicone | 2.00 |
| HallBrite ® BHB | Butyloctyl salicylate | 3.50 |
| Fragrance | Fragrance | 0.50 |

The sunscreen composition was prepared by heating the Phase B components to 60° C. while mixing. The Phase A components were separately mixed. The Phase B components were added to the Phase A components with mixing to produce a sprayable sunscreen composition.

The resulting composition had a static SPF of 60.3 and a post-immersion SPF of 57.8. Thus 95.85% of the composition's SPF rating was retained following immersion in water for 80 minutes. As can be seen from these results, inclusion of 2.0% by weight of Silsoft® 440 into the sunscreen composition resulted in a sunscreen composition that had good water retention properties and a post-immersion SPF of greater than 57.

### Example 2

In this example, a sunscreen composition comprising sunscreen actives, Silsoft® 440, a carrier, an emollient, and a fragrance, but no waterproofing agent was prepared and the static SPF, post immersion SPF, and % SPF retained following the water immersion test was determined according to the procedures described in the Test Methods section. The following components were used to prepare the composition:

TABLE 2

| | | |
|---|---|---|
| Trade Name | INCI Name | % weight |

Phase A

| | | |
|---|---|---|
| SD Alcohol-200 Proof | SD Alcohol 40B | 74.70 |

Phase B

| | | |
|---|---|---|
| Escalol ® 557 | Octinoxate | 7.50 |
| Escalol ® 587 | Octisalate | 5.00 |
| Escalol ® 597 | Octocrylene | 0.80 |
| Escalol ® 517 | Avobenzone | 1.00 |
| Escalol ® 567 | Oxybenzone | 5.00 |
| Silsoft ® 440 | PEG-20/PPG-23 dimethicone | 2.00 |
| HallBrite ® BHB | Butyloctyl salicylate | 3.5 |
| Fragrance | Fragrance | 0.5 |

The sunscreen composition was prepared as described in Example 1. The resulting composition had a static SPF of 47.7 and a post-immersion SPF of 39.7. Thus 83.23% of the composition's SPF rating was retained following immersion in water for 80 minutes. As can be seen from these results, inclusion of 2.0% by weight of Silsoft® 440 in the sunscreen composition resulted in a sunscreen composition that had good water retention properties, even in the absence of a hydrophobic waterproofing agent, such as Dermacryl® 79.

### Example 3

In this example, a sunscreen composition comprising sunscreen actives, Silsoft® 440, a carrier, an emollient, and a fragrance, but no waterproofing agent was prepared and the static SPF, post immersion SPF, and % SPF retained following the water immersion test was determined according to the procedures described in the Test Methods section. The following components were used to prepare the composition:

TABLE 3

| | | |
|---|---|---|
| Trade Name | INCI Name | % weight |

Phase A

| | | |
|---|---|---|
| SD Alcohol-200 Proof | SD Alcohol 40B | 72.70 |

Phase B

| | | |
|---|---|---|
| Escalol ® 557 | Octinoxate | 7.50 |
| Escalol ® 587 | Octisalate | 5.00 |
| Escalol ® 597 | Octocrylene | 0.80 |
| Escalol ® 517 | Avobenzone | 1.00 |
| Escalol ® 567 | Oxybenzone | 5.00 |
| Silsoft ® 440 | PEG-20/PPG-23 dimethicone | 4.00 |
| HallBrite ® BHB | Butyloctyl salicylate | 3.5 |
| Fragrance | Fragrance | 0.5 |

The sunscreen composition was prepared as described in Example 1. The resulting composition had a static SPF of 48.4 and a post-immersion SPF of 48.7. Thus 100% of the composition's SPF rating was retained following immersion in water for 80 minutes. As can be seen from these results, inclusion of 4.0% by weight of Silsoft® 440 and less than 20% by weight of sunscreen actives in the sunscreen composition resulted in a sunscreen composition that had good water retention properties and a post-immersion SPF of greater than 48, even in the absence of a hydrophobic waterproofing agent, such as Dermacrylo79.

### Example 4

In this example, a sunscreen composition comprising sunscreen actives, Silsoft® 440, a carrier, an emollient, a fragrance, and a waterproofing agent was prepared and the static SPF, post immersion SPF, and % SPF retained following the water immersion test was determined according to the procedures described in the Test Methods section. The following components were used to prepare the composition:

TABLE 4

| | | |
|---|---|---|
| Trade Name | INCI Name | % weight |

Phase A

| | | |
|---|---|---|
| SD Alcohol-200 Proof | SD Alcohol 40B | 73.70 |
| Dermacryl ® 79 | Acrylates/octylacrylamide | 2.00 |
| | copolymer | |

Phase B

| | | |
|---|---|---|
| Escalol ® 557 | Octinoxate | 7.50 |
| Escalol ® 587 | Octisalate | 5.00 |
| Escalol ® 597 | Octocrylene | 0.80 |
| Escalol ® 517 | Avobenzone | 1.00 |
| Escalol ® 567 | Oxybenzone | 5.00 |
| Silsoft ® 440 | PEG-20/PPG-23 dimethicone | 1.00 |
| HallBrite ® BHB | Butyloctyl salicylate | 3.5 |
| Fragrance | Fragrance | 0.5 |

The sunscreen composition was prepared as described in Example 1. The resulting composition had a static SPF of 63.2 and a post-immersion SPF of 60.1. Thus 95.09% of the composition's SPF rating was retained following immersion in water for 80 minutes. As can be seen from these results, lowering the amount of Silsoft® 440 to 1.0% by weight in combination with 2.0% by weight of a waterproofing agent and less than 20% by weight of sunscreen actives in the sunscreen composition resulted in a sunscreen composition that had good water retention properties and a post-immersion SPF of greater than 60.

### Example 5

In this example, a sunscreen composition comprising sunscreen actives, a PEG-3 dimethicone, a carrier, an emollient, a fragrance, and a waterproofing agent was prepared and the static SPF, post immersion SPF, and % SPF retained following the water immersion test was determined according to the procedures described in the Test Methods section. The following components were used to prepare the composition:

TABLE 5

| | | |
|---|---|---|
| Trade Name | INCI Name | % weight |

Phase A

| | | |
|---|---|---|
| SD Alcohol-200 Proof | SD Alcohol 40B | 72.70 |
| Dermacryl ® 79 | Acrylates/octylacrylamide | 2.00 |
| | copolymer | |

Phase B

| | | |
|---|---|---|
| Escalol ® 557 | Octinoxate | 7.50 |
| Escalol ® 587 | Octisalate | 5.00 |
| Escalol ® 597 | Octocrylene | 0.80 |
| Escalol ® 517 | Avobenzone | 1.00 |
| Escalol ® 567 | Oxybenzone | 5.00 |
| KF-945A Fluid | PEG-3 dimethicone | 2.00 |
| HallBrite ® BHB | Butyloctyl salicylate | 3.5 |
| Fragrance | Fragrance | 0.5 |

The sunscreen composition was prepared as described in Example 1. The resulting composition had a static SPF of 57.9 and a post-immersion SPF of 51.1. Thus 88.26% of the composition's SPF rating was retained following immersion in water for 80 minutes. As can be seen from these results, the PEG-3 dimethicone was effective at providing a sunscreen composition that had good water retention properties and a post-immersion SPF of greater than 51.

### Example 6

In this example, a sunscreen composition comprising sunscreen actives, a PEG-12 dimethicone, a carrier, an emollient, a fragrance, and a waterproofing agent was prepared and the static SPF, post immersion SPF, and % SPF retained following the water immersion test was determined according to the procedures described in the Test Methods section. The following components were used to prepare the composition:

TABLE 6

| | | |
|---|---|---|
| Trade Name | INCI Name | % weight |

Phase A

| | | |
|---|---|---|
| SD Alcohol-200 Proof | SD Alcohol 40B | 72.70 |
| Dermacryl ® 79 | Acrylates/octylacrylamide | 2.00 |
| | copolymer | |

Phase B

| | | |
|---|---|---|
| Escalol ® 557 | Octinoxate | 7.50 |
| Escalol ® 587 | Octisalate | 5.00 |
| Escalol ® 597 | Octocrylene | 0.80 |
| Escalol ® 517 | Avobenzone | 1.00 |
| Escalol ® 567 | Oxybenzone | 5.00 |
| DC 193 Fluid (Dow | PEG-12 dimethicone | 2.00 |
| Corning) | | |
| HallBrite ® BHB | Butyloctyl salicylate | 3.5 |
| Fragrance | Fragrance | 0.5 |

The sunscreen composition was prepared as described in Example 1. The resulting composition had a static SPF of 62.2 and a post-immersion SPF of 51.9. Thus 83.44% of the composition's SPF rating was retained following immersion in water for 80 minutes. As can be seen from these results, the PEG-12 dimethicone was effective at providing a sunscreen composition that had good water retention properties and a post-immersion SPF of greater than 51.

### Example 7

In this example, a sunscreen composition comprising sunscreen actives, a PPG-12 dimethicone, a carrier, an emollient, a fragrance, and a waterproofing agent was prepared and the static SPF, post immersion SPF, and % SPF retained following the water immersion test was determined according to the procedures described in the Test Methods section. The following components were used to prepare the composition:

TABLE 7

| | | |
|---|---|---|
| Trade Name | INCI Name | % weight |

Phase A

| | | |
|---|---|---|
| SD Alcohol-200 Proof | SD Alcohol 40B | 72.70 |
| Dermacryl ® 79 | Acrylates/octylacrylamide | 2.00 |
| | copolymer | |

Phase B

| | | |
|---|---|---|
| Escalol ® 557 | Octinoxate | 7.50 |
| Escalol ® 587 | Octisalate | 5.00 |
| Escalol ® 597 | Octocrylene | 0.80 |
| Escalol ® 517 | Avobenzone | 1.00 |
| Escalol ® 567 | Oxybenzone | 5.00 |
| Silsoft ® 920 | PPG-12 dimethicone | 2.00 |
| HallBrite ® BHB | Butyloctyl salicylate | 3.5 |
| Fragrance | Fragrance | 0.5 |

The sunscreen composition was prepared as described in Example 1. The resulting composition had a static SPF of 63.2 and a post-immersion SPF of 57.9. Thus 91.61% of the composition's SPF rating was retained following immersion in water for 80 minutes. As can be seen from these results, the PPG-12 dimethicone was effective at providing a sunscreen composition that had good water retention properties and a post-immersion SPF of greater than 57.

### Example 8

In this example, a sunscreen composition comprising sunscreen actives, a carrier, a waterproofing agent, an emollient, and a fragrance, but no dimethicone derivative was prepared and the static SPF, post-immersion SPF, and % SPF retained following the water immersion test was determined according to the procedures described in the Test Methods section. The following components were used to prepare the composition:

TABLE 8

| | | |
|---|---|---|
| Trade Name | INCI Name | % Wt |

Phase A

| | | |
|---|---|---|
| SD Alcohol-200 Proof | SD Alcohol 40B | 66.80 |
| Dermacryl ® 79 | Acrylates/octylacrylamide | 2.00 |
| | copolymer | |
| Dermacryl ® AQF | Acrylates copolymer | 4.40 |

Phase B

| | | |
|---|---|---|
| Escalol ® 557 | Octinoxate | 7.50 |
| Escalol ® 587 | Octisalate | 5.00 |
| Escalol ® 597 | Octocrylene | 0.80 |
| Escalol ® 517 | Avobenzone | 1.00 |
| Escalol ® 567 | Oxybenzone | 5.00 |
| Eusolex ® HMS | Homosalate | 3.50 |
| HallBrite ® BHB | Butyloctyl salicylate | 3.50 |
| Fragrance | Fragrance | 0.50 |

The sunscreen composition was prepared as described in Example 1. The resulting composition had a static SPF of 65.1 and a post-immersion SPF of 41.7. Thus 64.06% of the composition's SPF rating was retained following immersion in water for 80 minutes. As can be seen from these results, a sunscreen composition comprising the traditional waterproofing agents Dermacryl® 79 and Dermacryl® AQF, but no dimethicone derivative had a much lower % SPF retained following the water immersion test as compared to sunscreen compositions comprising a dimethicone derivative as disclosed herein.

### Example 9

In this example, a sunscreen composition comprising sunscreen actives, a carrier, an emollient, a fragrance, and a waterproofing agent, but no dimethicone derivative was prepared and the static SPF, post immersion SPF, and % SPF retained following the water immersion test was determined according to the procedures described in the Test Methods section. The following components were used to prepare the composition:

TABLE 9

| | | |
|---|---|---|
| Trade Name | INCI Name | % weight |

Phase A

| | | |
|---|---|---|
| SD Alcohol-200 Proof | SD Alcohol 40B | 72.70 |
| Dermacryl ® 79 | Acrylates/octylacrylamide | 4.00 |
| | copolymer | |

Phase B

| | | |
|---|---|---|
| Escalol ® 557 | Octinoxate | 7.50 |
| Escalol ® 587 | Octisalate | 5.00 |
| Escalol ® 597 | Octocrylene | 0.80 |
| Escalol ® 517 | Avobenzone | 1.00 |
| Escalol ® 567 | Oxybenzone | 5.00 |
| HallBrite ® BHB | Butyloctyl salicylate | 3.5 |
| Fragrance | Fragrance | 0.5 |

The sunscreen composition was prepared as described in Example 1. The resulting composition had a static SPF of 58.7 and a post-immersion SPF of 25.3. Thus only 43.10% of the composition's SPF rating was retained following immersion in water for 80 minutes. As can be seen from these results, a sunscreen composition comprising the traditional waterproofing agent Dermacryl® 79, but no dimethicone derivative had a much lower % SPF retained following the water immersion test and a much lower post-immersion SPF as compared to sunscreen compositions comprising the same amount of sunscreen actives but also comprising a dimethicone derivative as disclosed herein.

### Example 10

In this example, a sunscreen composition comprising sunscreen actives, a carrier, a fragrance, and a waterproofing agent, but no dimethicone derivative and no emollient was prepared and the static SPF, post immersion SPF, and % SPF retained following the water immersion test was determined according to the procedures described in the Test Methods section. The following components were used to prepare the composition:

TABLE 10

| | | |
|---|---|---|
| Trade Name | INCI Name | % weight |

Phase A

| | | |
|---|---|---|
| SD Alcohol-200 Proof | SD Alcohol 40B | 76.24 |
| Dermacryl ® AQF | Acrylates copolymer | 3.96 |

Phase B

| | | |
|---|---|---|
| Escalol ® 557 | Octinoxate | 7.50 |
| Escalol ® 587 | Octisalate | 5.00 |
| Escalol ® 597 | Octocrylene | 0.80 |
| Escalol ® 517 | Avobenzone | 1.00 |
| Escalol ® 567 | Oxybenzone | 5.00 |
| Fragrance | Fragrance | 0.5 |

The sunscreen composition was prepared as described in Example 1. The resulting composition had a static SPF of 53.0 and a post-immersion SPF of 39.1. Thus 73.77% of the composition's SPF rating was retained following immersion in water for 80 minutes. As can be seen from these results, a sunscreen composition comprising the traditional waterproofing agent Dermacryl® 79, but no dimethicone derivative and no emollient had a lower % SPF retained following the water immersion test and a lower post-immersion SPF as compared to sunscreen compositions comprising the same amount of sunscreen actives but also comprising a dimethicone derivative as disclosed herein.

### Examples 11 to 13

In this example, lotion based sunscreen compositions were prepared and the static SPF, post immersion SPF, and % SPF retained following the water immersion test were determined according to the procedures described in the Test Methods section. The following components were used to prepare the compositions:

TABLE 11

| | | | | |
|---|---|---|---|---|
| | | Ex. 11 | Ex. 12 | Ex. 13 |
| Trade Name | INCI Name | (wt %) | (wt %) | (wt %) |

Phase A

| | | | | |
|---|---|---|---|---|
| Water | Water | 61.68 | 60.68 | 61.68 |
| Pemulen ® TR-2 | Acrylates/C12-20 | 0.20 | 0.20 | 0.20 |
| | Acrylate Copolymer | | | |
| Chlorphenisen | Chlorphenisen | 0.30 | 0.30 | 0.30 |
| Methylparaben | Methylparaben | 0.16 | 0.16 | 0.16 |
| Propylparaben | Propylparaben | 0.16 | 0.16 | 0.16 |
| Glycerin | Glycerin | 1.00 | 1.00 | 1.00 |
| Avicel ® PC 611 | Microcrystalline | 1.00 | 1.00 | 1.00 |
| | Cellulose and Cellulose | | | |
| | Gum | | | |
| Versene ® Na2 | Disodium EDTA | 0.10 | 0.10 | 0.10 |
| (Dow) | | | | |

Phase B

| | | | | |
|---|---|---|---|---|
| Elefac ® I-205 | Octyldodecyl | 5.00 | 5.00 | 5.00 |
| | Neopentanoate | | | |
| Finsolv ® TN | C12-15 Alkyl Benzoate | 2.00 | 2.00 | 2.00 |
| Escalol ® 557 | Octinoxate | 7.50 | 7.50 | 7.50 |
| Escalol ® 587 | Octisalate | 5.00 | 5.00 | 5.00 |
| Escalol ® 597 | Octocrylene | 0.80 | 0.80 | 0.80 |
| Escalol ® 517 | Avobenzone | 1.00 | 1.00 | 1.00 |
| Escalol ® 567 | Oxybenzone | 5.00 | 5.00 | 5.00 |
| Lexgard ® O | Capryl Glycol | 0.75 | 0.75 | 0.75 |
| Amphisol ® A | Cetyl Phosphate | 1.00 | 1.00 | 1.00 |
| SunSpheres ® | Styrene/Acrylates | 1.00 | 1.00 | 1.00 |
| Powder | Copolymer | | | |

Phase C

| | | | | |
|---|---|---|---|---|
| Silsoft ® 440 | PEG-20/PPG-23 | 0 | 2.00 | 2.00 |
| | dimethicone | | | |
| Allianz ® OPT | Acrylates/C12-22 alkyl | 2.00 | 1.00 | 0 |
| | methacrylate copolymer | | | |
| Tocopheryl | Tocopheryl Acetate | 0.005 | 0.005 | 0.005 |
| acetate | | | | |
| Actiphyte of | — | 0.05 | 0.05 | 0.05 |
| Aloe Vera 10 | | | | |
| Fold | | | | |

Phase D

| | | | | |
|---|---|---|---|---|
| Paragon MEPB | Phenoxyethanol and | 1.00 | 1.00 | 1.00 |
| | Methylparaben and | | | |
| | Ethylparaben and | | | |
| | Propylparaben and | | | |
| | Butylparaben | | | |
| Fragrance | Fragrance | 0.30 | 0.30 | 0.30 |

Phase E

| | | | | |
|---|---|---|---|---|
| KOH (10%) | Potasium Hydroxide | 3.00 | 3.00 | 3.00 |

The sunscreen compositions were prepared by heating the Phase B components to 75-80° C. while mixing. The Phase A components were separately heated to 75-80° C. until the preservatives were dissolved, with the Pemulen® TR-2 being gradually added. While homogenizing the Phase A mixture, the Phase B mixture was added to Phase A with mixing at 4000-5000 rpm for two minutes. After two minutes, batch mixing was continued, and the mixture was gradually cooled. Once the mixture was below 50° C., the phase C ingredients were added. The resulting mixture was cooled, and the Phase D ingredients were added once the mixture was at or below 35° C. The pH of the final composition was adjusted to about 6.5 using the Phase E ingredient.

The static SPF, post immersion SPF, and % SPF retained following the water immersion test for each composition were measured as previously described. The results are given in Table 12.

TABLE 12

| Composition | Static SPF | Post-immersion SPF | % SPF retained |
|---|---|---|---|
| Example 11 | 53.8 | 46.5 | 86.43% |
| Example 12 | 61.9 | 61.2 | 98.87% |
| Example 13 | 62.5 | 59.2 | 94.72% |

As can be seen from these results, the compositions comprising the Silsoft® 440 dimethicone derivative (i.e., the Ex. 12 and Ex. 13 compositions) had much higher post-immersion SPF ratings than did the composition comprising the waterproofing agent (the Allianz® OPT) but no dimethicone derivative (i.e., the Ex. 11 composition). The Example 12 and 13 compositions also had better % SPF retained following the water immersion test than the Example 11 composition, which did not contain any dimethicone derivative. These results indicate that adding a dimethicone derivative as described herein to a lotion-based sunscreen formulation can improve the post-immersion SPF and % SPF retained following the water immersion test of sunscreen compositions as compared to sunscreen compositions comprising a waterproofing agent but no dimethicone derivative. Furthermore, the Example 12 composition, which comprised both the waterproofing agent and 2.0% by weight of the Silsoft® 440, had a higher post-immersion SPF and higher % SPF retained following the water immersion test than either of the compositions that comprised just the waterproofing agent or just the Silsoft® 440 dimethicone derivative.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved and other advantageous results attained.

As various changes could be made in the above compositions and products without departing from the scope of the disclosure, it is intended that all matter contained in the above description shall be interpreted as illustrative and not in a limiting sense.

What is claimed is:

1. A sunscreen composition comprising from about 38% (by total weight of the composition) to about 92% (by total weight of the composition) of a carrier, not more than about 25% (by total weight of the composition) of a sunscreen active, and from about 0.5% (by total weight of the composition) to about 5.0% (by total weight of the composition) of a dimethicone derivative selected from the group consisting of polyoxyethylene derivatized dimethicone, polyoxypropylene derivatized dimethicone, polyoxyethylene/polyoxypropylene derivatized dimethicone, and combinations thereof.

2. The sunscreen composition of claim 1 further comprising from about 0.5% (by total weight of the composition) to about 5% (by total weight of the composition) of a waterproofing agent.

3. The sunscreen composition of claim 2 wherein the waterproofing agent is selected from the group consisting of acrylates/octylacrylamide copolymer, acrylates copolymer, acrylates/C₁₂₋₂₂ alkyl methacrylate copolymer, and combinations thereof.

4. The sunscreen composition of claim 1 wherein the sunscreen active is selected from the group consisting of aminobenzoic acid, avobenzone, cinoxate, dioxybenzone, homosalate, menthyl anthranilate, octocrylene, octinoxate, octisalate, oxybenzone, padimate O, phenylbenzimidazole sulfonic acid, sulisobenzone, titanium dioxide, trolamine salicylate, zinc oxide, camphor benzalkonium methosulfate, terephthalidene dicamphor sulfonic acid, benzylidene camphor sulfonic acid, polyacrylamidomethyl benzylidene camphor, PEG-25 PABA isoamyl p-methoxycinnamate, ethylhexyl triazone, drometrizole trielloxane, diethylhexyl butamido triazone, 4-methylbenzylidene camphor, 3-benzylidene camphor, ethylhexyl dimethyl PABA, methylene bis-benztriazolyl tetramethylbutylphenol, disodium phenyl dibenzimidazole tetrasulfonate, bis-ethylhexyloxyphenol methoxyphenol triazine, methylene bisbenzotriazolyl tetramethylbutylphenol, bisethylhexyloxyphenol methoxyphenyl triazine, and combinations thereof.

5. The sunscreen composition of claim 4 wherein the sunscreen active is selected from the group consisting of avobenzone, octocrylene, octinoxate, octisalate, oxybenzone, homosalate, and combinations thereof.

6. The sunscreen composition of claim 5 wherein the composition comprises about 1.0% (by total weight of the composition) of avobenzone, about 7.5% (by total weight of the composition) octinoxate, about 5.0% (by total weight of the composition) oxybenzone, about 5.0% (by total weight of the composition) octisalate, about 0.8% (by total weight of the composition) octocrylene, and about 4.0% (by total weight of the composition) homosalate.

7. The sunscreen composition of claim 1 wherein the polyoxyethylene derivatized dimethicone is selected from the group consisting of PEG-1 dimethicone, PEG-3 dimethicone, PEG-4 dimethicone, PEG-6 dimethicone, PEG-7 dimethicone, PEG-8 dimethicone, PEG-9 dimetyhicone, PEG-10 dimethicone, PEG-12 dimethicone, PEG-14 dimethicone, PEG-17 dimethicone, bis-PEG-20 dimethicone, and combinations thereof.

8. The sunscreen composition of claim 1 wherein the polyoxypropylene derivatized dimethicone is selected from the group consisting of PPG-2 dimethicone, PPG-12 dimethicone, PPG-27 dimethicone, PPG-12 butyl ether dimethicone, and combinations thereof.

9. The sunscreen composition of claim 1 wherein the polyoxyethylene/polyoxypropylene derivatized dimethicone, is selected from the group consisting of PEG-20/PPG-23 dimethicone, PEG/PPG 20-15 dimethicone, PEG-23/PPG-6 dimethicone, PPG-4 oleth-10 dimethicone, and combinations thereof.

10. The sunscreen composition of claim 9 wherein the polyoxyethylene/polyoxypropylene derivatized dimethicone is a PEG-20/PPG-23 dimethicone.

11. The sunscreen composition of claim 10 wherein the PEG-20/PPG-23 dimethicone has a molecular weight of about 20,000.

12. The sunscreen composition of claim 1 wherein the carrier is selected from the group consisting of alcohols, water, volatile ethers, and combinations thereof.

13. The sunscreen composition of claim 12 wherein the carrier is an alcohol selected from the group consisting of ethyl alcohol, isopropyl alcohol, methyl alcohol, propyl alcohol, butyl alcohol, ethylene glycol, propylene glycol, butylene glycol, pentylene glycol, hexylene glycol, dipropylene glycol, ethoxydiglycol, and combinations thereof.

14. The sunscreen composition of claim 1 further comprising an additional component selected from the group consisting of an emollient, a fragrance, a preservative, an antioxidant, a humectant, a rheology modifier, a light scattering or reflecting agent, an aesthetic modifier, and combinations thereof.

15. The sunscreen composition of claim 14 wherein the composition comprises not more than about 10.0% (by total weight of the composition) of an emollient, and the emollient is butyloctyl salicylate.

16. The sunscreen composition of claim 1 wherein the composition comprises about 1.0% (by total weight of the composition) of avobenzone, about 7.5% (by total weight of the composition) octinoxate, about 5.0% (by total weight of the composition) oxybenzone, about 5.0% (by total weight of the composition) octisalate, about 0.8% (by total weight of the composition) octocrylene, about 4.0% (by total weight of the composition) homosalate, from about 59.7% (by total weight of the composition) to about 73.7% (by total weight of the composition) of an alcohol, from about 1.0% (by total weight of the composition) to about 5.0% (by total weight of the composition) of an acrylates/octylacrylamide copolymer, from about 1.0% (by total weight of the composition) to about 5.0% (by total weight of the composition) of a PEG-20/PPG-23 dimethicone, from about 1.0% (by total weight of the composition) to about 5.0% (by total weight of the composition) of butyloctyl salicylate, and from about 0.5% (by total weight of the composition) to about 1.0% (by total weight of the composition) of fragrance.

17. The sunscreen composition of claim 1 wherein the composition comprises not more than about 20% (by total weight of the composition) of sunscreen active.

18. The sunscreen composition of claim 17 wherein the composition has a static SPF of at least about 40.

19. The sunscreen composition of claim 18 wherein the composition has a static SPF of at least about 50.

20. The sunscreen composition of claim 17 wherein the composition has a post-immersion SPF of at least about 40.

21. The sunscreen composition of claim 1 wherein the composition has a post-immersion SPF of at least about 50.

22. A sunscreen composition comprising a carrier, a sunscreen active, and a dimethicone derivative selected from the group consisting of polyoxyethylene derivatized dimethicone, polyoxypropylene derivatized dimethicone, polyoxyethylene/polyoxypropylene derivatized dimethicone, and combinations thereof, wherein from about 75% to about 100% of an SPF rating of the composition is retained following immersion in water for 80 minutes.

23. The sunscreen composition of claim 22 further comprising a waterproofing agent.

24. The sunscreen composition of claim 22 wherein the composition has a post-immersion SPF of at least about 40.

25. The sunscreen composition of claim 24 wherein the composition has a post-immersion SPF of at least about 50.
